(19) United States
(12) Patent Application Publication
(10) Pub. No.: US 2017/0367869 A1
(43) Pub. Date: Dec. 28, 2017

(54) METHOD AND APPARATUS FOR TREATING SCOLIOSIS

(71) Applicants: Allen Carl, Slingerlands, NY (US); William Fredenburg, Albany, NY (US)

(72) Inventors: Allen Carl, Slingerlands, NY (US); William Fredenburg, Albany, NY (US)

(21) Appl. No.: 15/524,714

(22) PCT Filed: Dec. 14, 2015
(86) PCT No.: PCT/US15/65550

§ 371 Date: May 5, 2017

Related U.S. Application Data
(60) Provisional application No. 62/090,937, filed on Dec. 12, 2014.

(51) Int. Cl. A61F 5/02

(52) U.S. Cl. CPC A61F 5/02

(57) ABSTRACT

A brace for treating scoliosis, the brace comprising: a lower portion for disposition about the hips of a patient; an upper portion for disposition about the thorax of a patient; a first support rod adjustably extending between the lower portion and the upper portion, the first support rod being disposed at least in part along one of the front side of the patient and the back side of the patient; a second support rod adjustably extending between the lower portion and the upper portion, the second support rod being disposed at least in part along one side of the patient; wherein the lower portion, the upper portion, the first support rod and the second sup port rod are configured such that (i) the upper portion may be adjustably fixedly set substantially parallel to the lower portion, and (ii) the upper portion may be adjustably fixedly set canted at an angle to the lower portion.

### REFERENCE TO PENDING PRIOR PATENT APPLICATION

This patent application claims benefit of pending prior U.S. Provisional Patent Application Ser. No. 62/090,937, filed Dec. 12, 2014 by Allen Carl et al. for METHOD AND APPARATUS FOR TREATING SCOLIOSIS (Attorney's Docket No. CARL-3A PROV), which patent application is hereby incorporated herein by reference.

### FIELD OF THE INVENTION

This invention relates to anatomical braces in general, and more particularly to braces for treating scoliosis.

### BACKGROUND OF THE INVENTION

Scoliosis is a medical condition in which a person's spine is curved laterally. As a result, when viewed from the rear, the person's spine may exhibit a curved and/or twisted configuration rather than a straight and aligned (i.e., with the longitudinal axis of the body) configuration. See, for example, FIGS. 1 and 2, which show a curved and/or twisted spine S which is typical for a person exhibiting scoliosis.

Where the scoliosis is modest, the quality of the patient's life may not be significantly affected and treatment may consist primarily of observation. However, where the scoliosis is substantial, the quality of the patient's life may be significantly impacted, e.g., the patient may exhibit substantial physical deformity, may have difficulty walking, standing and/or sitting, may suffer excessive fatigue of the spine's supporting musculature, may experience structural pain, etc. Orthopedic spinal bracing is frequently prescribed for patients whose scoliosis is progressing or suggests future complications and reductions in quality of life. In extreme cases, surgical intervention may be required, however, such surgery is generally complex and often results in reduced mobility of the spine.

Current orthopedic braces generally fall into two categories, i.e., they are either a “full time” support brace (sometimes also referred to as a “day brace” or an “18 hour day brace”) for providing a modest level of support for the patient while the patient is awake or asleep, or an “overcorrection” brace for providing an aggressive level of overcorrection (e.g., while the patient is supine, generally while sleeping). Current orthopedic braces are designed to provide one or the other of these bracing therapies, but not both. Patient compliance (and therapeutic outcome) may vary depending upon the type of brace selected. Thus, patients must generally purchase two different braces in order to obtain both bracing therapies, i.e., a “full time” support brace and an “overcorrection” brace. Since a scoliosis brace typically costs approximately $1500.00-$4000.00, the need to purchase two separate braces in order to obtain both bracing therapies represents a substantial financial burden, particularly inasmuch as insurance reimbursements have been reduced.

Thus there is a need for a new and improved scoliosis brace which is capable of functioning as both a “full time” support brace and an “overcorrection” brace, thereby providing the benefit of both bracing therapies in a single brace.

### SUMMARY OF THE INVENTION

This and other objects of the present invention are addressed by the provision and use of a new method and apparatus for treating scoliosis.

Among other things, the present invention provides a new and improved scoliosis brace which is capable of functioning as both a “full time” support brace and an “overcorrection” brace, thereby providing both bracing therapies in a single brace.

In one preferred form of the invention, there is provided a brace for treating scoliosis, the brace comprising:

a lower portion for disposition about the hips of a patient;

an upper portion for disposition about the thorax of a patient;

a first support rod adjustably extending between the lower portion and the upper portion, the first support rod being disposed at least in part along one of the front side of the patient and the back side of the patient;

a second support rod adjustably extending between the lower portion and the upper portion, the second support rod being disposed at least in part along one side of the patient;

wherein the lower portion, the upper portion, the first support rod and the second support rod are configured such that (i) the upper portion may be adjustably fixedly set substantially parallel to the lower portion, and (ii) the upper portion may be adjustably fixedly set canted at an angle to the lower portion.

In another preferred form of the invention, there is provided a method for treating scoliosis, the method comprising:

providing a brace for treating scoliosis, the brace comprising:
a lower portion for disposition about the hips of a patient;
an upper portion for disposition about the thorax of a patient;
a first support rod adjustably extending between the lower portion and the upper portion, the first support rod being disposed at least in part along one of the front side of the patient and the back side of the patient;
a second support rod adjustably extending between the lower portion and the upper portion, the second support rod being disposed at least in part along one side of the patient;
wherein the lower portion, the upper portion, the first support rod and the second support rod are configured such that (i) the upper portion may be adjustably fixedly set substantially parallel to the lower portion, and (ii) the upper portion may be adjustably fixedly set canted at an angle to the lower portion;

positioning the brace on the patient;

adjustably fixedly setting the upper portion substantially parallel to the lower portion; and

thereafter adjustably fixedly setting the upper portion canted at an angle to the lower portion.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1 and 2 are exemplary views showing scoliosis in a patient;

FIG. 3 is a front schematic view showing a novel scoliosis brace formed in accordance with the present invention;

FIGS. 4 and 5 are front and side schematic views, respectively, showing further aspects of the scoliosis brace shown in FIG. 3;

FIG. 6 is a front schematic view showing the novel scoliosis brace of FIG. 3 being used in its “overcorrection” brace position;

FIG. 7 is a front schematic view showing a modified form of the novel scoliosis brace of FIG. 3 being used in its “full time” support brace position;

FIG. 8 is a front schematic view showing the novel scoliosis brace of FIG. 7 being used in its “overcorrection” brace position; and

FIGS. 9-12 are schematic views showing another novel scoliosis brace formed in accordance with the present invention.

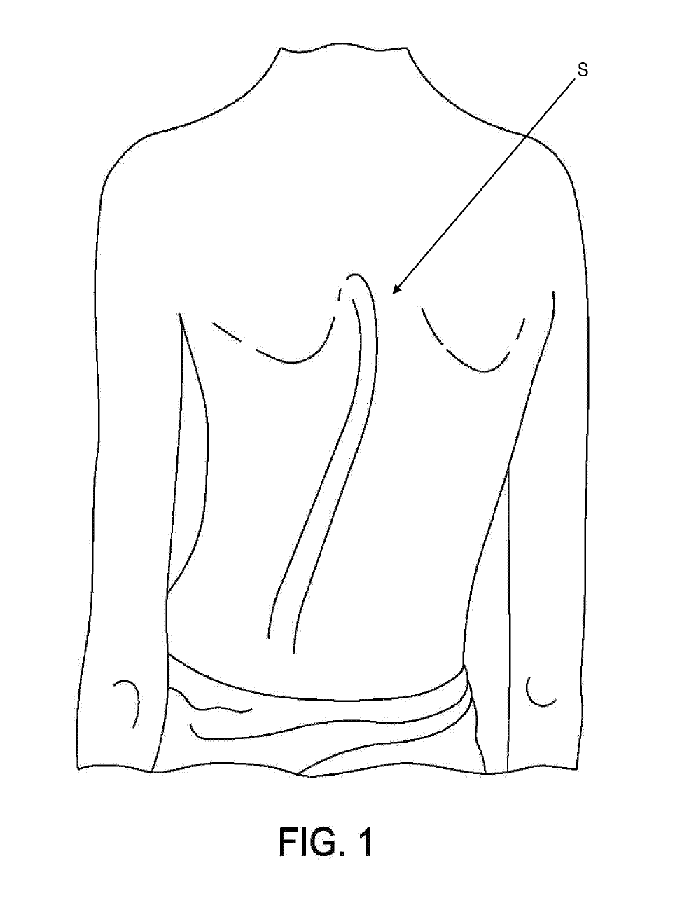

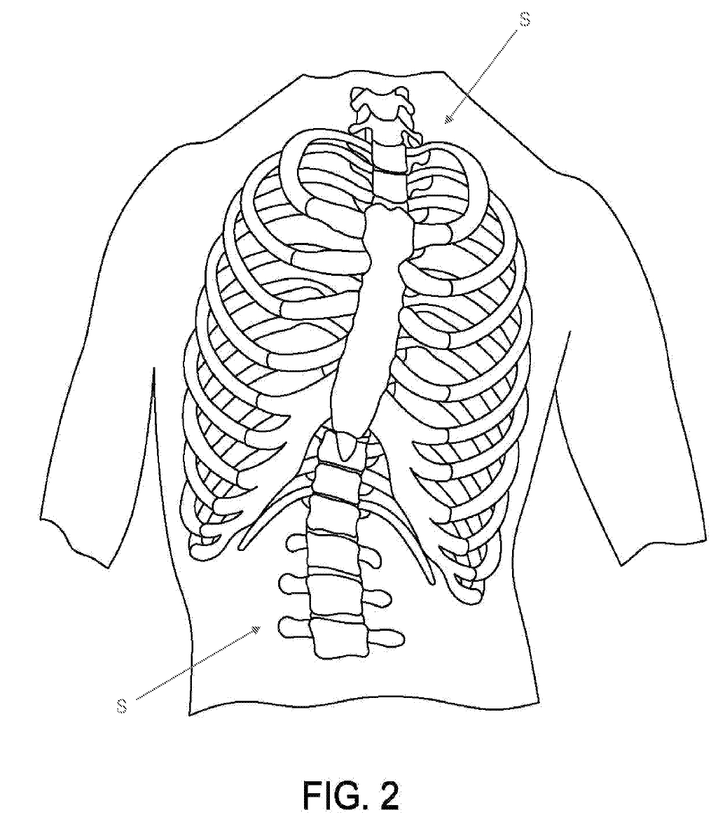

### DETAILED DESCRIPTION OF THE PREFERRED EMBODIMENTS

The present invention provides a new and improved scoliosis brace which is capable of functioning as both a “full time” support brace and an “overcorrection” brace, thereby providing both bracing therapies in a single brace.

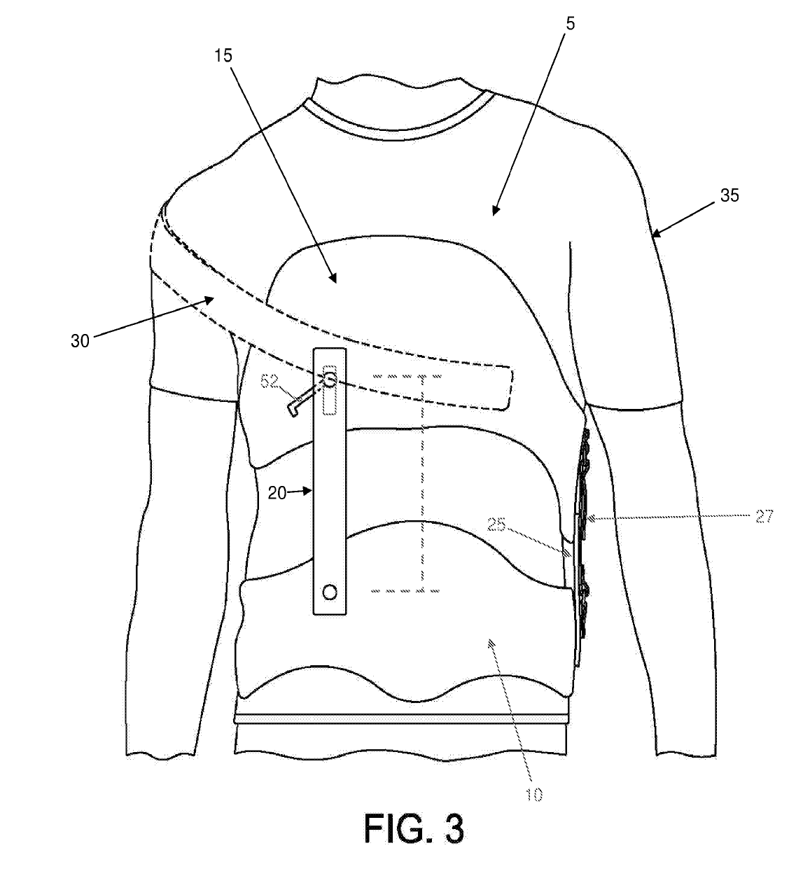

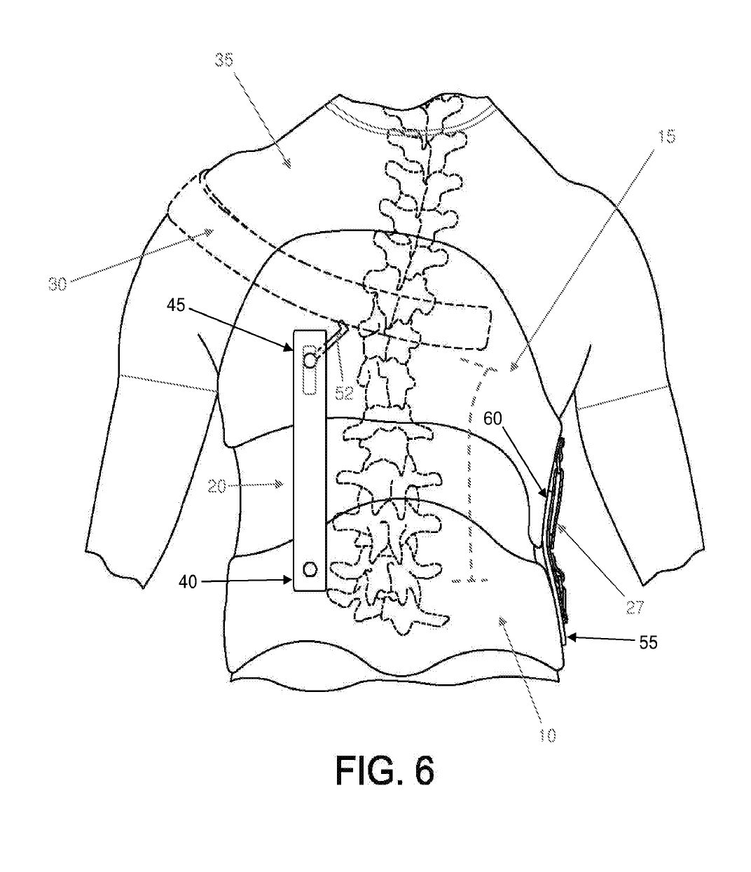

More particularly, and looking now at FIGS. 3-6, there is shown a novel scoliosis brace 5 formed in accordance with the present invention and which is capable of functioning as both a “full time” support brace and an “overcorrection” brace. FIGS. 3-5 show the novel scoliosis brace in its “full time” support brace position. FIG. 6 shows the novel scoliosis brace in its “overcorrection” brace position.

More particularly, the novel scoliosis brace 5 generally comprises a lower portion 10 for positioning about the hips of the patient; an upper portion 15 for positioning about the thorax of the patient; a front rod 20 for adjustably connecting upper portion 15 of scoliosis brace 5 to lower portion 10 of scoliosis brace 5; and a side rod 25 for adjustably connecting upper portion 15 of scoliosis brace 5 to lower portion 10 of scoliosis brace 5. The disposition of upper portion 15 of scoliosis brace 5 is adjusted vis-à-vis the disposition of lower portion 10 of scoliosis brace 5 in order to apply the desired force vectors to the patient's anatomy, as will hereinafter be discussed in further detail.

In one preferred form of the invention, scoliosis brace 5 also comprises a cinching mechanism 27 for selectively applying forces to upper portion 15 of scoliosis brace 5 vis-à-vis lower portion 10 of scoliosis brace 5, and for restricting movement of upper portion 15 of scoliosis brace 5 relative to lower portion 10 of scoliosis brace 5, so as to facilitate functioning of scoliosis brace 5, particularly when scoliosis brace 5 is in its “overcorrection” position.

One or more straps 30 may also be used to secure upper portion 15 of scoliosis brace 5 to the patient, and/or to secure lower portion 10 of scoliosis brace 5 to the patient, and/or to connect upper portion 15 of scoliosis brace 5 to lower portion 10 of scoliosis brace 5. Straps 30 allow further “on the fly” adjustment of the force vectors which are applied to the patient's anatomy by scoliosis brace 5.

If desired, padding (not shown) may be introduced between the patient's body and lower portion 10 of scoliosis brace 5, and/or between the patient's body and upper portion 15 of scoliosis brace 5. This padding allows further adjustment of the force vectors being applied to the patient's anatomy, whereby to allow for controlling and re-aligning of the deformity of spine S with the desired anatomical alignment.

In one form of the invention, novel scoliosis brace 5 may also comprise a “velcro” shirt 35 which is worn over the body of the patient and beneath scoliosis brace 5. This “velcro” shirt allows one or more of lower portion 10 of scoliosis brace 5, upper portion 15 of scoliosis brace 5, strap(s) 30 and/or the aforementioned padding to adhere to the “velcro” shirt, whereby to further stabilize and further re-align the elements of the scoliosis deformity (including higher up relative to scoliosis brace 5) relative to the patient's anatomy.

Lower portion 10 of scoliosis brace 5 is preferably formed out of a stiff material (e.g., molded plastic, carbon fiber, a composite material, etc.). Lower portion 10 may comprise two halves (e.g., a front half and a back half) which are secured together about the patient's anatomy so as to form the complete lower portion 10 of scoliosis brace 5. Alternatively, lower portion 10 of scoliosis brace 5 may comprise a single piece of material which is slit vertically and which is flexible enough to allow lower portion 10 of scoliosis brace 5 to be temporarily forced apart for fitting over the patient's anatomy and thereafter grasps the patient's anatomy in an acceptable fashion.

Upper portion 15 of scoliosis brace 5 is also preferably formed out of a stiff material (e.g., molded plastic, carbon fiber, a composite material, etc.). Upper portion 15 may also comprise two halves (e.g., a front half and a back half) which are secured together about the patient's anatomy so as to form the complete upper portion 15 of scoliosis brace 5. Alternatively, upper portion 15 of scoliosis brace 5 may comprise a single piece of material which is slit vertically and which is flexible enough to allow upper portion 15 of scoliosis brace 5 to be temporarily forced apart for fitting over the patient's anatomy and thereafter grasps the patient's anatomy in an acceptable fashion.

Front rod 20 adjustably connects upper portion 15 of scoliosis brace 5 to lower portion 10 of scoliosis brace 5 so as to allow upper portion 15 of scoliosis brace 5 to be set a desired distance from lower portion 10 of scoliosis brace 5, and so as to allow upper portion 15 of scoliosis brace 5 to be inclined (or “tilted”) at a desired angle relative to lower portion 10 of scoliosis brace 5. More particularly, in one preferred form of the invention, front rod 20 comprises a bottom end 40 which is fixed to lower portion 10 of scoliosis brace 5, and a top end 45 which is adjustably received in a front mount 50 provided in upper portion 15 of scoliosis brace 5. This front mount 50 allows the upper portion 15 of scoliosis brace 5 to be selectively fixed in various positions vis-à-vis the top end 45 of front rod 20. More particularly, front mount 50 is constructed so that upper portion 15 of scoliosis brace 5 can be selectively moved up or down, and/or tilted, relative to lower portion 10 of scoliosis brace 5 by selectively adjusting the connection between front mount 50 and the top end 45 of front rod 20, and thereafter securing top end 45 of front rod 20 to front mount 50 (i.e., when upper portion 15 of scoliosis brace 5 is disposed at the desired height and at the desired angle relative to lower portion 10 of scoliosis brace 5). As a result of this construction, upper portion 15 of scoliosis brace 5 can be set the desired distance from lower portion 10 of scoliosis brace 5, and upper portion 15 of scoliosis brace 5 can be set at the desired angle of tilt relative to lower portion 10 of scoliosis brace 5.

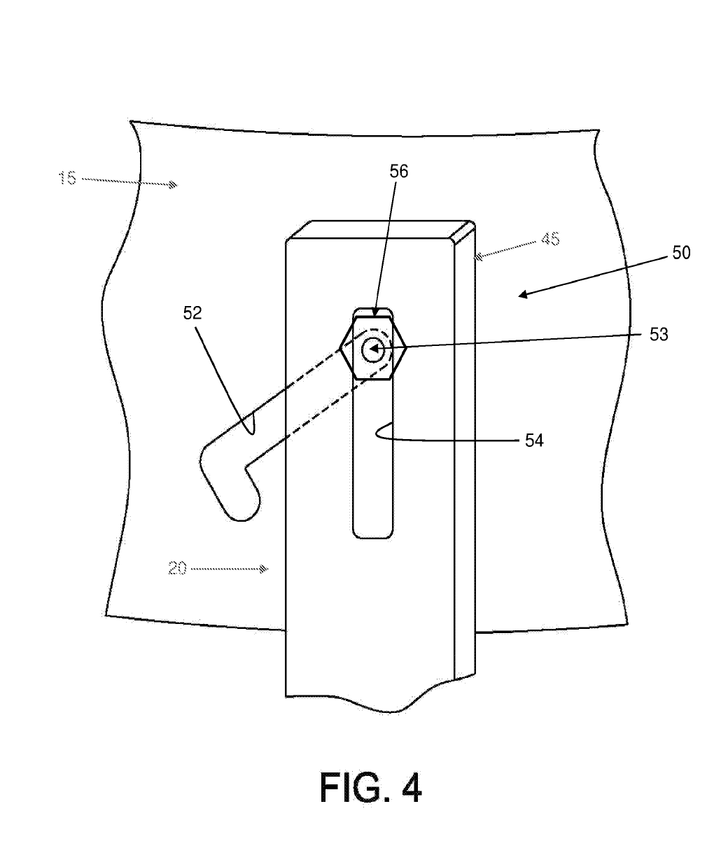

In one preferred form of the invention, front mount 50 may comprise an inclined slot 52, with top end 45 of front rod 20 being slidably mounted to inclined slot 52 via a screw-and-washer mechanism which may be loosened/tightened as desired. By way of example but not limitation, a bolt 53 may extend through inclined slot 52 formed in upper portion 15 and through a slot (or hole or holes) 54 formed in top end 45 of front rod 40. A nut 56 may be secured over bolt 53, whereby to lock upper portion 15 to front rod 40 in a desired position. See FIG. 4. If desired, inclined slot 52 may be replaced with a pattern or template of holes, or one or more oversized openings, etc., i.e., so as to allow for alignment variability by choosing the holes or openings used for securing upper portion 15 to front rod 20.

Side rod 25 also adjustably connects upper portion 15 of scoliosis brace 5 to lower portion 10 of scoliosis brace 5. More particularly, in one preferred form of the invention, side rod 25 comprises a bottom end 55 which is fixed to lower portion 10 of scoliosis brace 5, and a top end 60 which is adjustably slidably received in a side mount 65 provided in upper portion 15 of scoliosis brace 5. This side mount 65 allows the upper portion 15 of scoliosis brace 5 to be selectively fixed in various positions vis-à-vis top end 60 of side rod 25. More particularly, side mount 65 is constructed so that upper portion 15 of scoliosis brace 5 can be selectively moved up and down relative to lower portion 10 of scoliosis brace 5, by selectively adjusting the connection between side mount 65 and the top end 60 of side rod 25, and thereafter securing top end 60 of side rod 25 to side mount 65 (i.e., when upper portion 15 of scoliosis brace 5 is disposed in the desired position relative to lower portion 10 of scoliosis brace 5).

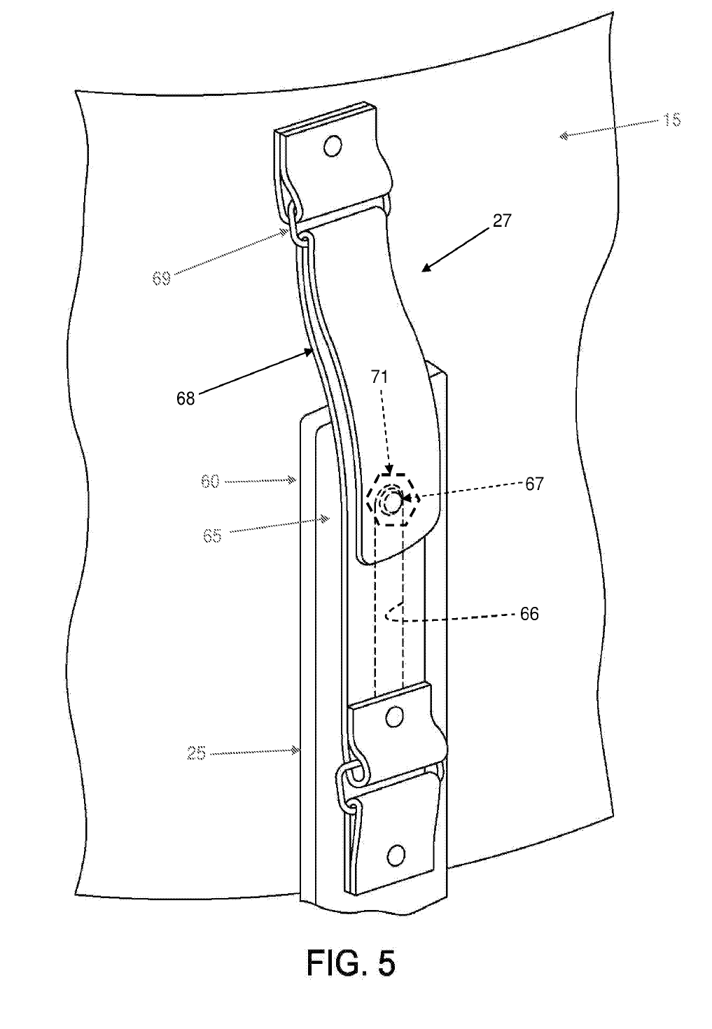

In one preferred form of the invention, side mount 65 may comprise a slot 66 formed in side rod 25 for receiving a protrusion (e.g., a bolt) 67 which is secured to upper portion 15. Protrusion 67 is sized to slide within slot 66 when upper portion 15 of scoliosis brace 5 is moved up or down relative to lower portion 10 of scoliosis brace 5. And in a preferred form of the present invention, a nut 71 is provided which may be secured over protrusion (e.g., a bolt) 67, whereby to lock upper portion 15 to side rod 25 in a desired position. See FIG. 5.

In one preferred form of the invention, a cinching mechanism 27 is provided to selectively apply forces to upper portion 15 of scoliosis brace 5 vis-à-vis lower portion 10 of scoliosis brace 5, and for restricting movement of upper portion 15 of scoliosis brace 5 relative to lower portion 10 of scoliosis brace 5, whereby to facilitate functioning of scoliosis brace 5, particularly when scoliosis brace 5 is in its “overcorrection” position. Cinching mechanism 27 generally comprises a length of material 68 which extends between upper portion 15 of scoliosis brace 5 and lower portion 10 of scoliosis brace 5 and which may be shortened as desired, whereby to selectively apply forces to upper portion 15 of scoliosis brace 5 and to restrict movement of the upper portion 15 of scoliosis brace 5 relative to lower portion 10 of scoliosis brace 5.

In one preferred form of the invention, cinching mechanism 67 comprises a length of web 68 which extends between upper portion 15 of scoliosis brace 5 and lower portion 10 of scoliosis brace 5, and which includes a tightening buckle 69 which allows the length of web 68 to be shortened as desired. By way of example but not limitation, tightening buckle 69 may comprise a cinching buckle which can releasably lock web 68 under tension, or tightening buckle 69 may comprise a sliding buckle and web 68 may be locked to itself (e.g., with a Velcro mechanism, etc.).

On account of the foregoing construction, when scoliosis brace 5 is to be used as a “full time” support brace for providing a modest level of deformity support/correction to the patient (e.g., while the patient is awake or asleep), upper portion 15 of scoliosis brace 5 is positioned the appropriate distance from lower portion 10 of scoliosis brace 5, with upper portion 15 of scoliosis brace 5 positioned substantially parallel to lower portion 10 of scoliosis brace 5, and front rod 20 is secured in mount 50 and side rod 25 is secured in side mount 65. See FIG. 3. When scoliosis brace 5 is in this configuration, front rod 20 will be secured “higher up” in front mount 50 and side rod 25 will be secured “lower down” in side mount 65. In one preferred form of the invention, cinching mechanism 27 is used to restrict movement of upper portion 15 of scoliosis brace 5 relative to lower portion 10 of scoliosis brace 5 when the scoliosis brace is disposed in the proper position. And in one preferred form of the present invention, “velcro” fitted shirt 35 is worn by the patient so as to be disposed between the body of the patient and scoliosis brace 5, and the “velcro” fitted shirt 35 is used to further adjust the scoliosis deformity and stabilize it to the upper portion 15 of scoliosis brace 5 relative to lower portion 10 of scoliosis brace 5. In addition, one or more straps 30 may also be used to adjust the position of the upper portion 15 of scoliosis brace 5 relative to lower portion 15 of scoliosis brace 5, so as to adjust the force vectors applied to the patient's body from various positions and/or heights. Alternatively and/or additionally, padding may be introduced between the patient's body and lower portion 10 of scoliosis brace 5 and/or between the patient's body and upper portion 15 of scoliosis brace 5, so as to adjust the force vectors applied to the patient's body from various positions and/or heights.

When scoliosis brace 5 is to be used as an “overcorrection” brace for providing an aggressive level of overcorrection (e.g., while the patient is in a supine position such as when sleeping), upper portion 15 of scoliosis brace 5 is repositioned so that it is set at an angle (i.e., pivoted) relative to lower portion 10 of scoliosis brace 5 (i.e., upper portion 15 is canted clockwise, off of the horizontal, when viewed from the frame of reference of FIGS. 3 and 6), whereby to apply an overcorrective force to the anatomy of the patient. See FIG. 6. When this occurs, front rod 20 is secured “lower down” in front mount 50 and side rod 25 is secured “higher up” in side mount 65. In one preferred form of the invention, cinching mechanism 27 is used to apply forces to upper portion 15 as needed, and is then set so as to restrict movement of upper portion 15 relative to lower portion 10 when scoliosis brace 5 is disposed in the proper position. In one preferred form of the invention, “velcro” shirt 35 is worn by the patient underneath scoliosis brace 5, and “velcro” fitted shirt 35 may be used to further stabilize upper portion 15 of scoliosis brace 5 relative to lower portion 10 of scoliosis brace 5 and to further stabilize the higher scoliotic curve relative to scoliosis brace 5. In addition, one or more straps 30 may also be used to adjust the position upper portion 15 of scoliosis brace 5 relative to lower portion 15 of scoliosis brace 5 and to further stabilize the scoliotic curvature relative to scoliosis brace 5, so as to adjust the force vectors applied to the patient's body from various positions and/or heights. Alternatively and/or additionally, padding may be introduced between the patient's body and lower portion 10 of scoliosis brace 5 and/or between the patient's body and upper portion 15 of scoliosis brace 5, so as to adjust the force vectors applied to the patient's body.

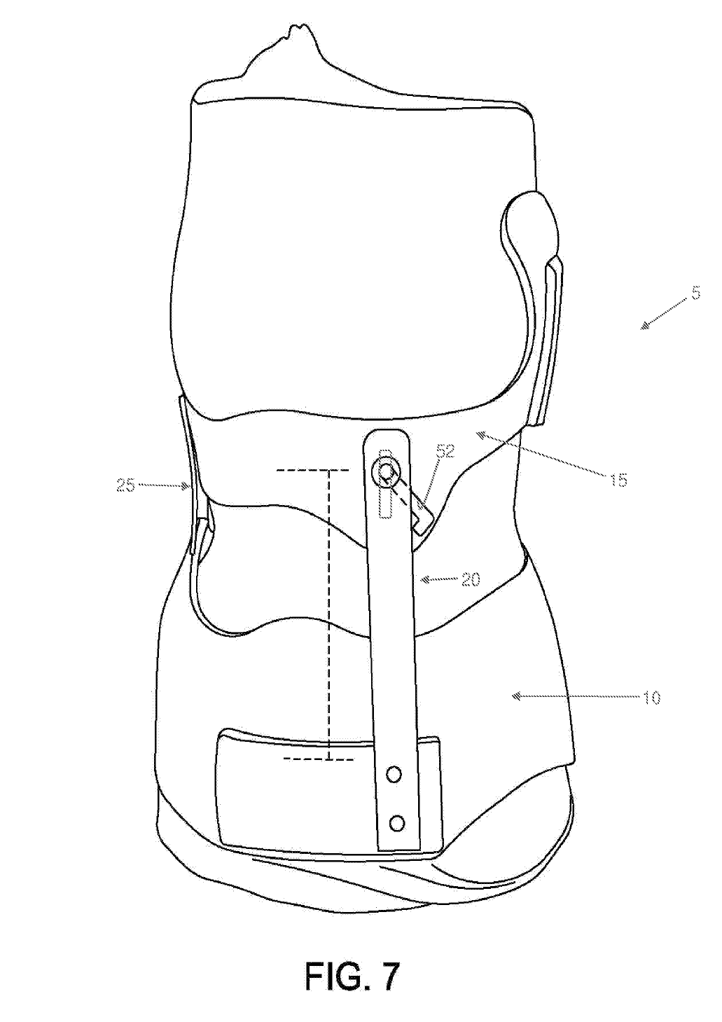

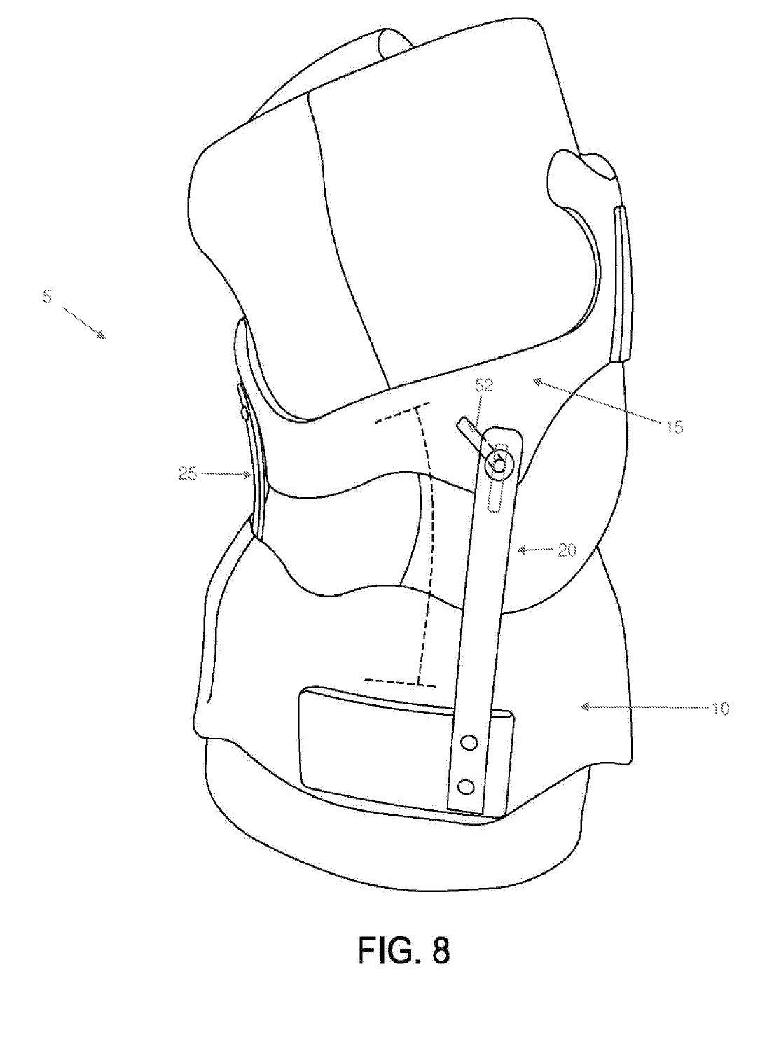

It will be appreciated that upper portion 15 of scoliosis brace 5 and lower portion 10 of scoliosis brace 5 may be provided in various shapes and/or sizes in order to contour to the anatomy of a given patient. See for example, FIGS. 7 and 8, which show further exemplary configurations for the upper portion of the scoliosis brace and the lower portion of the scoliosis brace.

It will also be appreciated that front rod 20 of scoliosis brace 5 may be set on either the left side of the torso, the right side of the torso or, less preferably, the center of the torso. See, for example, FIGS. 7 and 8, which show front rod 20 set on the opposite side of the body from the position shown in FIGS. 3-6. Front rod 20 can also be configured to cant the brace in a way that rotates, derotates or untwists the spine between the upper and lower attachment points. Furthermore, front rod 20 of scoliosis brace 5 may be set on the back side of the body.

It will also be appreciated that side rod 25 of scoliosis brace 5 may be set on either the left side of the torso or the right side of the torso. See, for example, FIGS. 7 and 8, which show side rod 25 set on the opposite side of the body from the position shown in FIGS. 3-6.

And it will be appreciated that cinching mechanism 27 may be set on either the left side of the torso or the right side of the torso, although it will generally be set on the side of the torso to which upper portion 15 of scoliosis brace 5 will be tilted when scoliosis brace 5 is set in its “overcorrection” position (since positioning cinching mechanism 27 on this side of the torso allows it to “pull down” on upper portion 15 of scoliosis brace 5 and thereby apply the desired forces to the body when scoliosis brace 5 is to be used in its “overcorrection” position).

And it will be appreciated that front rod 20 of scoliosis brace 5, front mount 50, side rod 25 and/or side mount 65 of scoliosis brace 5 may take various configurations consistent with the present invention.

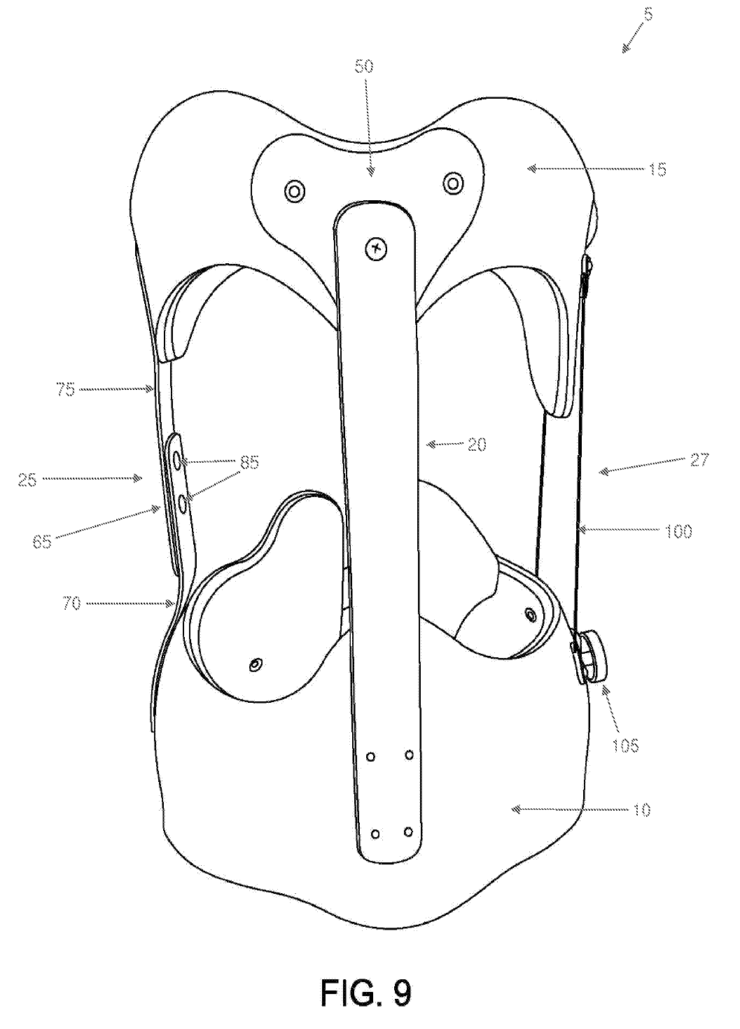

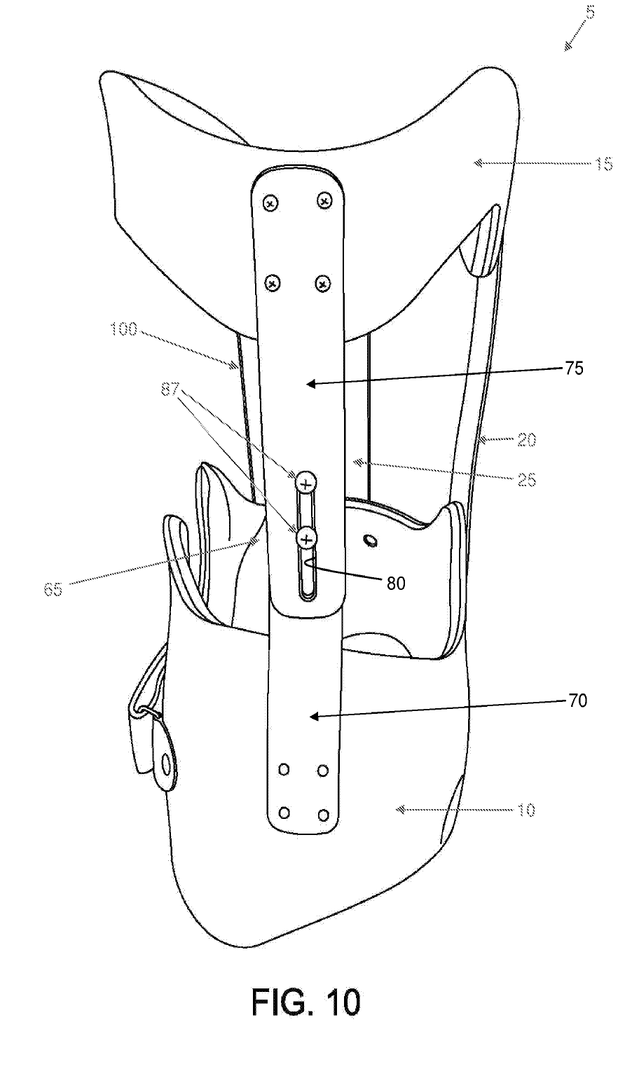

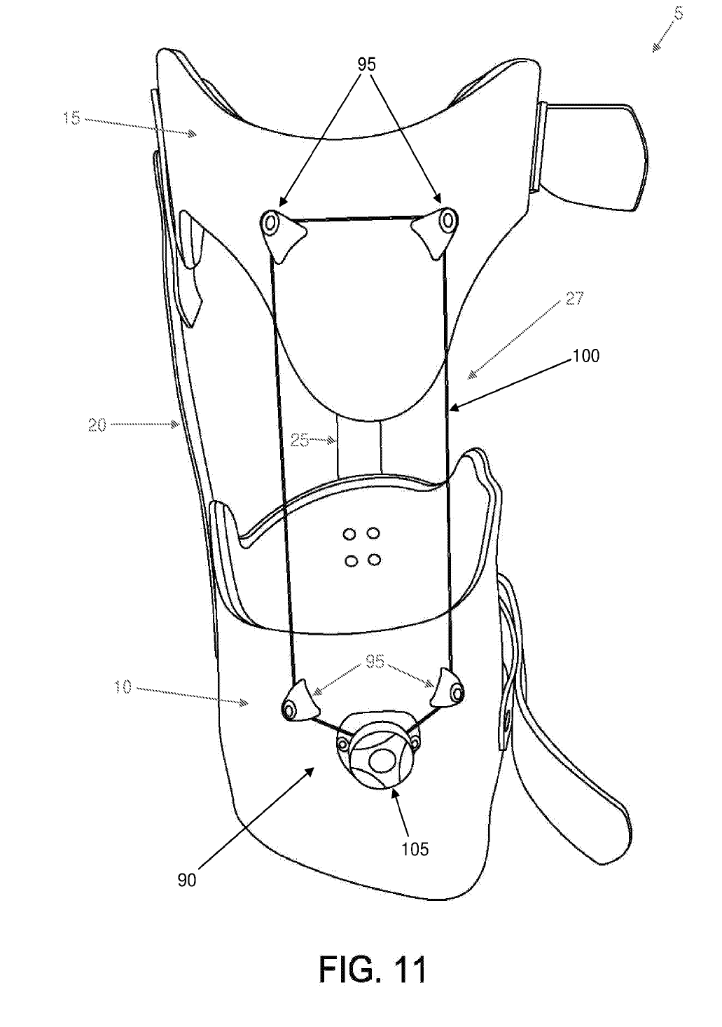

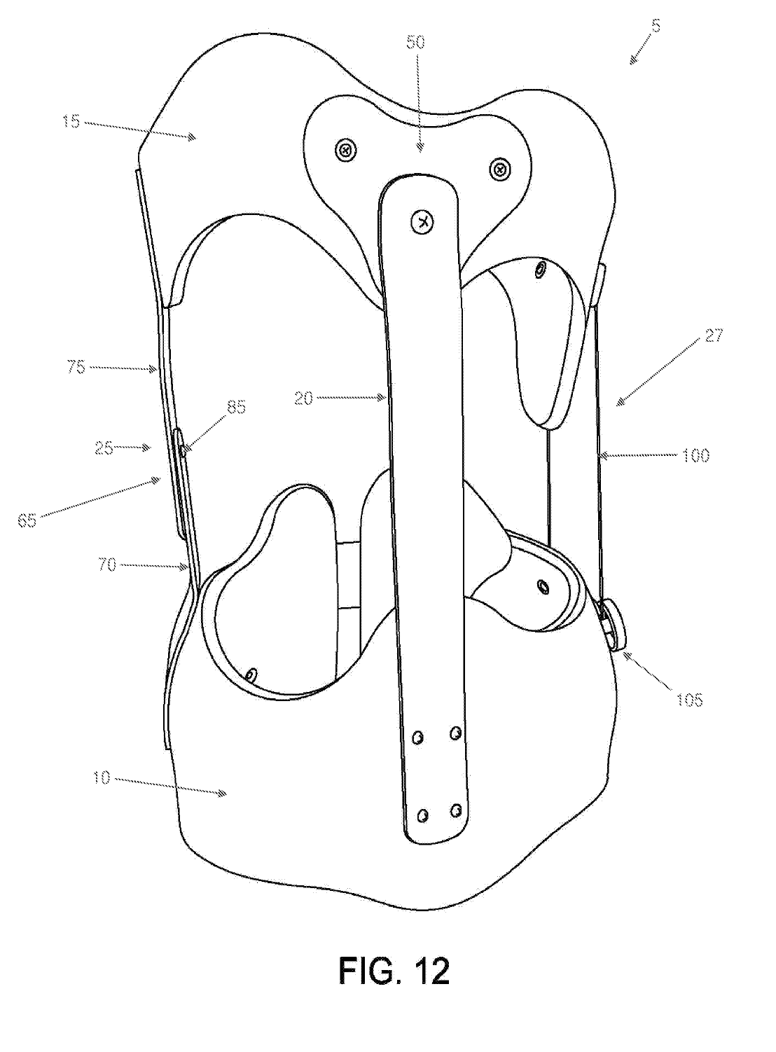

By way of example but not limitation, in another preferred form of the present invention, and looking now at FIGS. 9-12, there is shown a novel scoliosis brace 5 wherein upper portion 15 is mounted to lower portion 10 by way of a centrally-located front rod 20 and a centrally-located front mount 50. If desired, front mount 50 may comprise a slot, or a pattern or template of holes, or one or more oversized openings, etc., i.e., so as to allow for alignment variability by choosing the holes or openings used for securing upper portion 15 to front rod 20. Side rod 25 comprises a lower half 70 fixedly mounted to lower portion 10, and an upper half 75 fixedly mounted to upper portion 15, with upper half 75 being slidably mounted to a lower half 70. Upper half 75 of side rod 25 comprises a slot 80 for receiving one or more protrusions (e.g., a bolt) 85 which are secured to lower half 70 of side rod 25 (e.g., with a nut 87), such that protrusions 85 slidably connect upper half 75 to lower half 70, whereby to form the aforementioned side mount 65. Cinching mechanism 27 comprises a cable tensioning mechanism 90 which is preferably mounted opposite side rod 25. Cable tensioning mechanism 90 preferably comprises a plurality of mounting points 95 for receiving a cable loop 100, as will hereinafter be discussed in greater detail. Mounting points 95 are preferably mounted to both upper portion 15 of scoliosis brace 5 and lower portion 10 of scoliosis brace 5. A tensioning knob 105 is mounted to lower portion 10 of scoliosis brace 5 (FIG. 11). Cable loop 100 is passed through the plurality of mounting points 95 and engages tensioning knob 105 such that cable loop 100 extends between lower portion 10 of scoliosis brace 5 and upper portion 15 of scoliosis brace 5. Cable loop 100 engages tensioning knob 105 such that rotation of tensioning knob 105 causes cable loop 100 to be effectively shortened, whereby to pull upper portion 15 of scoliosis brace 5 towards lower portion 10 of scoliosis brace 5, whereby to selectively and controllably pivot upper portion 15 of scoliosis brace 5 relative to lower portion 10 of scoliosis brace 5. By virtue of this construction, upper portion 15 of scoliosis brace 5 can be pivoted about the point where front rod 20 is mounted to upper portion 15 (i.e., front mount 50) and upper half 70 of side rod 25 can slide relative to lower half 75 of side rod 25 (i.e., within side mount 65). It will be appreciated that front mount 50, side mount 65 and cinching mechanism 27 are locked in position when scoliosis brace 5 is disposed in the desired position.

It should be appreciated that, if desired, upper portion 15 can be made to twist relative to lower portion 10 so as to apply torsion forces to the torso of the patient.

To this end, if desired, the mounting points 95 discussed above can be located so as to apply torsional forces to upper portion 15 (and hence to the torso of a patient) when cable loop 100 is shortened, as will hereinafter be discussed in greater detail. More particularly, the locations of mounting points 95 can be tailored to induce the desired torsional forces on upper portion 15 of scoliosis brace 5 so as to maximize the therapeutic benefit of scoliosis brace 5. By way of example but not limitation, mounting points 95 can be mounted to upper portion 15 “off center” (i.e., relative to tensioning knob 105) and/or to the front or back of upper portion 15 of scoliosis brace 5 so as to “twist” upper portion 15 of scoliosis brace 5 relative to lower portion 10 of scoliosis brace 5 when cable loop 100 is shortened by rotating tensioning knob 105. In other words, by varying the location of mounting points 95, upper portion 15 of scoliosis brace 5 can be caused to both pivot (off the horizontal) and twist (circumferentially) relative to lower portion 10 of scoliosis brace 5, thereby increasing the range of therapeutic options available when using scoliosis brace 5.

Furthermore, as discussed above, in one form of the present invention, front rod 20 is slidably mounted to upper portion 15 by way of a bolt-and-nut mechanism. More particularly, bolt 53 extends through the inclined slot 52 formed in upper portion 15 and through a slot (or hole) 54 formed in top end 45 of front rod 20 and a nut 56 is provided to lock upper portion 15 to front rod 25 in a desired position. Where this construction is utilized, one way of applying torsion forces to the torso of a patient is by canting inclined slot 52 relative to the plane of front mount 50, such that movement of the bolt within the canted inclined slot 52 causes upper portion 15 to twist (circumferentially) relative to lower portion 10.

Similarly, as discussed above, side rod 25 is slidably mounted to upper portion 15 by way of a protrusion 67 which is slidably received in slot 66 formed in side rod 25. More particularly, protrusion (i.e., a bolt) 67 extends through a hole formed in upper portion 15 such that it extends outwardly from upper portion 15 and is slidably received in slot 66 formed in side rod 25 and nut 71 is provided, which may be secured over protrusion 67, whereby to lock upper portion 15 to side rod 25 in a desired position. Where this construction is utilized, another way of applying torsion forces to the torso of a patient is by canting slot 66 relative to the plane of side mount 65, such that movement of protrusion 67 within the canted slot 66 causes upper portion 15 to twist relative to lower portion 10.

Torsion can also be applied to the torso of the patient by varying the shape/geometry of one or both of front rod 20 and side rod 25. By way of example but not limitation, front rod 20 and/or side rod 25 may be twisted so as to induce torsion forces on the patient's torso. Alternatively, front rod 20 and/or side rod 25 may incorporate a bend so as to induce torsion forces on the patient's torso.

Additionally, it should further be appreciated that torsion forces may be imposed on the patient's torso by varying the mounting points of cinching mechanism 27, e.g., by mounting one end of cinching mechanism 27 to the front or back of upper portion 15 and mounting the other end of cinching mechanism 27 to the side of lower portion 10 (or, optionally, by mounting one end of cinching mechanism 27 to the side of upper portion 15 and mounting the other end of cinching mechanism 27 to the front or back of lower portion 10). By virtue of this construction, when cinching mechanism 27 is tightened, upper portion 15 can be made to twist (circumferentially) relative to lower portion 10.

And it should also be appreciated that upper portion 15 may be mounted to front rod 20 of scoliosis brace 5 via a bolt (or bolts) disposed in one or more holes formed in upper portion 15 of scoliosis brace 5, or via a bolt disposed in a sliding hole or slot (e.g., such as the slot 52 shown in FIGS. 3, 4 and 6). To this end, upper portion 15 can comprise a “template” having a plurality of holes, slots, sliding holes, etc., in order to provide various options for mounting upper portion 15 to front rod 20 of scoliosis brace 5.

Furthermore, it should also be appreciated that upper portion 15 of scoliosis brace 5 and lower portion 10 of scoliosis brace 5 may be provided in different sizes/shapes/geometries in order to accommodate the specific anatomy of a given patient. Upper portion 15 of scoliosis brace 5 and lower portion 10 of scoliosis brace 5 may be provided “off the shelf” in common sizes/shapes/geometries, or upper portion 15 of scoliosis brace 5 and lower portion 10 of scoliosis brace 5 may be custom-fitted to a given patient or even custom designed and generated (e.g., via 3D printing).

### Modifications

It should also be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A brace for treating scoliosis, the brace comprising:
a lower portion for disposition about the hips of a patient;
an upper portion for disposition about the thorax of a patient;
a first support rod adjustably extending between the lower portion and the upper portion, the first support rod being disposed at least in part along one of the front side of the patient and the back side of the patient;
a second support rod adjustably extending between the lower portion and the upper portion, the second support rod being disposed at least in part along one side of the patient;
wherein the lower portion, the upper portion, the first support rod and the second support rod are configured such that (i) the upper portion may be adjustably fixedly set substantially parallel to the lower portion, and (ii) the upper portion may be adjustably fixedly set canted at an angle to the lower portion.

2. A brace for treating scoliosis according to claim 1 wherein the upper portion is pivotally adjustably mounted to the first support rod.

3. A brace for treating scoliosis according to claim 1 wherein the first support rod is disposed to one side of center.

4. A brace for treating scoliosis according to claim 1 wherein the first support rod is disposed on center.

5. A brace for treating scoliosis according to claim 1 wherein the upper portion is variably adjustably mounted to the second support rod.

6. A brace for treating scoliosis according to claim 1 wherein the second support rod is disposed at least in part on the left side of the patient.

7. A brace for treating scoliosis according to claim 1 wherein the second support rod is disposed at least in part on the right side of the patient.

8. A brace for treating scoliosis according to claim 1 wherein the brace further comprises a cinching mechanism connected to the lower portion and the upper portion for applying forces to the upper portion vis-à-vis the lower portion and for limiting the distance between the upper portion and the lower portion, the cinching mechanism extending at least in part along one side of the patient.

9. A brace for treating scoliosis according to claim 8 wherein the cinching mechanism also extends along at least one of the front of the patient and the back of the patient, so as to apply torsion to the patient.

10. A method for treating scoliosis, the method comprising:
providing a brace for treating scoliosis, the brace comprising:
a lower portion for disposition about the hips of a patient;
an upper portion for disposition about the thorax of a patient;
a first support rod adjustably extending between the lower portion and the upper portion, the first support rod being disposed at least in part along one of the front side of the patient and the back side of the patient;
a second support rod adjustably extending between the lower portion and the upper portion, the second support rod being disposed at least in part along one side of the patient;
wherein the lower portion, the upper portion, the first support rod and the second support rod are configured such that (i) the upper portion may be adjustably fixedly set substantially parallel to the lower portion, and (ii) the upper portion may be adjustably fixedly set canted at an angle to the lower portion;
positioning the brace on the patient;
adjustably fixedly setting the upper portion substantially parallel to the lower portion; and
thereafter adjustably fixedly setting the upper portion canted at an angle to the lower portion.

11. A method according to claim 10 wherein the upper portion is pivotally adjustably mounted to the first support rod.

12. A method according to claim 10 wherein the first support rod is disposed to one side of center.

13. A method according to claim 10 wherein the first support rod is disposed on center.

14. A method according to claim 10 wherein the upper portion is variably adjustably mounted to the second support rod.

15. A method according to claim 10 wherein the second support rod is disposed at least in part on the left side of the patient.

16. A method according to claim 10 wherein the second support rod is disposed at least in part on the right side of the patient.

17. A method according to claim 10 wherein the brace further comprises a cinching mechanism connected to the lower portion and the upper portion for applying forces to the upper portion vis-à-vis the lower portion and for limiting the distance between the upper portion and the lower portion, the cinching mechanism extending along at least in part one side of the patient.

18. A method according to claim 17 wherein the cinching mechanism also extends along at least one of the front of the patient and the back of the patient, so as to apply torsion to the patient.
